(19) United States
(12) Patent Application Publication
(10) Pub. No.: US 2017/0202180 A1
(43) Pub. Date: Jul. 20, 2017

(54) A SELF LEARNING SYSTEM FOR IDENTIFYING STATUS AND LOCATION OF PET ANIMALS

(71) Applicant: GEOSATIS SA, Le Noirmont (CH)

(72) Inventor: Yu YANG, Cupertino, CA (US)

(73) Assignee: GEOSATIS SA, Le Noirmont (CH)

(21) Appl. No.: 15/324,941

(22) PCT Filed: Jul. 13, 2015
(86) PCT No.: PCT/US15/40167

§ 371 Date: Jan. 9, 2017

Related U.S. Application Data
(60) Provisional application No. 62/023,829, filed on Jul. 12, 2014.

(51) Int. Cl. A01K 11/00; H04W 52/02; H04W 4/02; A01K 29/00; G08B 29/18

(52) U.S. Cl. CPC A01K 11/008; A01K 29/005; G08B 29/186; G08B 29/188; H04W 4/026; H04W 52/0254

(57) ABSTRACT

A pet animal-wearable self learning device for has been disclosed. The device determines the status of the pet animal by analyzing wireless signal information gathered by the device over a predetermined period of time. The signal information is analyzed to identify useful patterns for determining the status of a pet animal. The pet animal status typically is a multi-dimensional, qualitative or quantitative indicator such as GPS Latitude/Longitude of the pet animal, safe or unsafe condition of the pet animal The status of the pet animal is determined by various factors/conditions such as identifying whether the pet animal is in an enclosed space or not, identifying if the pet is in extreme temperature not suitable for the pet animal's health, identifying whether the pet animal is moving or stationary, identifying if the pet-wearable device has been removed.

### CROSS REFERENCE TO BELATED: PATENT APPLICATIONS

The present application is a PCT application claiming priority of a U.S. provisional application No. 62/023,829 on 12 Jul. 2014 with the title “A DEVICE FOR SELF-EARNING PET STATUS BY TRACKING SURROUNDING WIRELESS SIGNAL”, the entire contents of which are incorporated herein by reference in its entirety.

### BACKGROUND

Technical Field

The embodiments herein relates to systems and methods used for iterative tracking of pet animals. Particularly, the embodiments herein relates to devices used for tracking the location of pet animals, and for gathering information about the surroundings of pet animals.

Description of the Related Art

Pet animals are like close friends to human beings and pet owners consider them members of their families. Close attention of pet animals is difficult or infeasible and pet owners are often concerned about the location of their pet animals and whether they are in a safe area. Off-the-shelf wearable products are used to track pet animal location using an embedded GPS receiver. The GPS location is taken at a pre-defined frequency, which is sometimes adjustable based on some rudimentary rules. Most products also use cellular connectivity to report pet animal location to a central server when it moves outside a pre-defined geo-fence. This fixed frequency report regardless of the exact pet animal status leads to waste of energy. As GPS and cellular operations consume significant amount of energy, minimizing GPS and cellular activities is critical to conserve battery power in such a wearable device.

A variety of pet animal tracking devices are available in the market. However, such devices require battery re-charge every few days, which obviously decreases the ease-of-use of such devices. More importantly, this reduces the residue battery capacity critical in finding a lost pet, which severely impacts the effectiveness of the products.

Hence, there exists a need for a pet-wearable device to track, locate as well as identify the status of a pet (whether the pet's safe or unsafe) while simultaneously minimizing the battery drain. There is also a need for a pet-wearable device with a cloud server and a mobile phone app, for determining pet status by leveraging wireless signal information gathered by the device over time. Further, there is also a need for a pet-wearable device capable of broadcasting distress signal to the owner, to help the owner to recover the pet, in case the pet is lost.

The above mentioned shortcomings, disadvantages and problems are addressed herein and the proposed solution will be understood by reading and studying the following specification.

### OBJECTIVES OF THE EMBODIMENTS

The primary object of the embodiments herein is to provide a pet-wearable device for determining the status of the pet by leveraging wireless signal information gathered by the device over time.

Another object of the embodiments herein is to provide a pet-wearable device, which uses the status of the pet to direct the usage of GPS and cellular techniques to minimize their energy consumption.

Yet another object of the embodiments herein is to provide a pet-wearable device with a Bluetooth and Wi-Fi radios to autonomously learn and infer pet status in order to smartly defer the use of GPS and cellular techniques until pet has a high probability to be unsafe.

Yet another object of the embodiments herein is to provide a pet-wearable device capable of broadcasting distress signal to the owner, to help the owner to recover the pet, in case the pet is lost.

Yet another object of the embodiments herein is to provide a pet-wearable device with a cloud server and a mobile phone app, for determining pet status by leveraging wireless signal information gathered by the device over time.

Yet another object of the embodiments herein is to develop an accurate model of signal patterns correlated to pet status, as well as temporal and spatial patterns of such sources by learning the received wireless signals over time.

These and other objects and advantages envisaged by the present disclosure will become readily apparent from the following detailed description taken in conjunction with the accompanying drawings.

### SUMMARY

The various embodiments herein envisages a self learning monitoring device for monitoring the status of pet animals wearing said device. The device (referred to as pet animal-wearable device leverages wireless signal information gathered over a period of time. The time series of the signal information is analyzed to identify useful patterns that are used to determine the status of the pet animal. The pet animal status is a multi-dimensional, qualitative or quantitative indicator such as but not limited to a GPS Latitude/Longitude of the pet animal, safe or unsafe condition of the pet animal, etc. The safe condition is any of a condition selected from the group comprising, pet animal being at home, or easily reachable by the owner (or owner's friends and family members), or at a place that Is confirmed by the owner to be safe. Examples of safe status include, the pet animal is on a regular walk with the owner or a designated pet animal walker; or the pet animal is at a pet animal hotel confirmed by the owner. Further, the unsafe condition refers to a condition where the pet animal is at an unknown or inaccessible place for the owner.

In accordance with the embodiments herein, the status of the pet animal is determined by various factors/conditions such as but not limited to identifying whether the pet animal is in an enclosed space or not, identifying if the pet animal is in extreme temperature not suitable for the pet animal's health, identifying whether the pet animal is moving or stationary, identifying if the pet animal-wearable device/tag is removed, etc.

In accordance with the embodiments herein, the determined status of the pet animal is used for smartly deferring the use of GPS and cellular techniques until pet animal has a high probability to be unsafe.

In accordance with the embodiments herein, the pet animal-wearable device comprises: a plurality of radio components such as but not limited to a Bluetooth radio, a Wi-Fi radio, an embedded GPS, a cellular communication module, etc; a plurality of sensors such as but not limited to a light sensor, a temperature sensor, a motion sensor, an accelerometer sensor, a gyroscope, etc; a central processing unit comprising a micro-controller and a flash memory; and a power management module comprising a battery and a battery charging circuit.

In accordance with the embodiments herein, the Bluetooth radio and Wi-Fi radio of the pet animal-wearable device are used to autonomously learn and infer pet animal status in order to smartly defer the use of GPS and the cellular communication module until pet animal has a high probability to be unsafe (e.g., far away from the home not accompanied by a family member or friend of the owner). Such deferred use of GPS and cellular communication module is critical in conserving the battery power of the pet animal-wearable device.

In accordance with, the embodiments herein, the Bluetooth radio and the Wi-Fi radio periodically receive Bluetooth and Wi-Fi signals respectively around the pet animal, with a much lower power consumption compared to the GPS and cellular communication module. Information including MAC address of the signal source and the signal strength, are extracted from the received signal. Further, the extracted information is stored in the flash memory of pet animal-wearable device with a timestamp.

In accordance with the embodiments herein, the pet animal-wearable device transmits the received Bluetooth and Wi-Fi signals from the Bluetooth radio and the Wi-Fi radio to a cloud server. Further, the pet animal-wearable device along with the cloud server analyzes the collected/extracted signals (Bluetooth and Wi-Fi signals) to identify signal sources that are highly correlated to the pet animal status.

In accordance with the embodiments herein, the light sensor is adapted for identifying whether the pet animal is in an enclosed space or not; the temperature sensor is adapted for identifying if the pet animal is in extreme temperature not suitable for the pet animal's health; the motion sensor is adapted for identifying whether the pet animal is moving or stationary; the accelerometer sensor is adapted for detecting magnitude and direction of pet animal's movement; and the gyroscope is adapted for measuring or maintaining orientation. Further, the output of the said plurality of sensors is used by the micro-controller of the central processing unit for determining the status of the pet animal.

In accordance with the embodiments herein, the Wi-Fi radio identifies residential or business Wi-Fi access points (APs), as they broadcast beacons. Further, the first three octets in a MAC address are used as the Organizational Unique Identifier (OUI). Based on the OUI, the Wi-Fi radio identifies popular brand names such as but not limited to IBM™ PCs, Samsung™ mobile phones add Apple™ tablets and smart phones.

In accordance with the embodiments herein, the Bluetooth and Wi-Fi radios autonomously learn and infer pet animal status. The method for learning the pet animal status comprises receiving signals from the owner's mobile device, which is usually strong (for example, stronger than −65 dBm in the measurement of Relative Signal Strength Indicator (RSSI)) when received and correlating the received signals to repeat pet animal related daily events, such as pet animal walk in the morning/evening and close proximity with the pet animal after dinner, receiving signals from the mobile devices owned by other family members (and anyone else who lives at home), which are usually strong when they appear and correlating the received signal to the pet animal being at home or on a walk with a family member, receiving signals from the mobile devices owned by a designated pet animal walker, which are usually strong when they appear and correlating the received signal to the pet animal on a walk, receiving signal from the home Wi-Fi access point, which is usually strong and appears whenever the pet animal is at home, receiving signals from the Wi-Fi access points in the close neighborhood, which are relatively weak (for example, weaker than −65 dBm in the measurement of Relative Signal Strength Indicator (RSSI)) but still appear most of the time when the pet animal is at home or in close proximity of the home such as in the yard, receiving signals from the Wi-Fi access points m the broad neighborhood, which may appear a few times every day and often follows a rough temporal order during pet animal walks, receiving and storing signals from the mobile devices that show up randomly at home (such as from visitors), and receiving signal from mobile devices that shows up regularly during a pet animal walk (such as from a friend or pet animal pal), and receiving signals from places occasionally visited by the pet animal, such as but not limited to shopping malls, veterinary clinics, pet animal hotels, and friends' home.

In accordance with the embodiments herein, a mobile application is installed in owner's/user's mobile phone for receiving user confirmation about the status of the pet animal wearing the pet animal wearable device. The user confirmation (e.g., via a mobile phone app) is required to confirm the pet animal is safe. Further, the GPS Latitude/Longitude information is used to identify the type of the place and its surrounding signal footprint. The identified place and its surrounding signal footprint information are stored into the flash memory of the pet animal-wearable device and/or in the cloud server. The identified place and its surrounding signal footprint information are used to determine the location and status of the pet animal.

In accordance with the embodiments herein, the central processing unit of the pet animal-wearable device learns the identified signals and the signal sources to infer or predict the pet animal status. Examples for pet animal status prediction include: (i) a regular walk is correlated to the disappearance of home and close neighbors' Wi-Fi AP and appearance of Wi-Fi APs in the broad neighborhood. Most of the time, the pet animal is constantly accompanied by the owner's mobile phone. The pet animal may also be accompanied by a family member's mobile phone or a designated pet animal walker's mobile phone. (ii) Accompanied by the owner's mobile but also observing many new signal sources indicating an occasional walk away from the neighborhood, e.g., walking in the mall or playing in a pet animal park. Again, pet animal may be accompanied in this case by a family member or a pet animal walker's mobile phone. (iii) The pet animal is dropped to a veterinary physician or pet animal hotel that has been previously visited. The device will recognize the surrounding wireless signal footprint to infer the type of the place. Sometimes, a GPS location is considered to further confirm the pet animal is indeed at a place indicated by the signal footprint. More specific and contextual questions are sent to the user/owner for confirmation, such as “Is your pet animal at this Veterinary physician?” via SMS or through a dedicated mobile application installed in the user's/owner's mobile phone.

In accordance with the embodiments herein, the above learning and analysis process iterates over time to develop an accurate model of signal patterns correlated to pet animal status.

In accordance with the embodiments herein, the pet animal-wearable devices upload data/information to a cloud server using Wi-Fi of Bluetooth connections. The cloud server aggregates data/information from a large population of pet animals to build a database of Wi-Fi APs that can be used to quickly infer pet animal status. For example, signal footprint of regular pet animal walkers in an area (such, as parks, veterinary physicians and pet animal hotels, etc) can be disseminated to the pet animal-wearable devices in the vicinity, so that the pet animal-wearable devices recognize this pet animal walker in future. Similar option applies to signal footprint of regional Veterinary physicians and pet animal hotels.

In accordance with the embodiments herein, the cloud server requests a geographic location server such as the Google™ Geo-location server for getting the GPS latitude/longitude information based on the Wi-Fi APs, to determine the location and status of the pet animal.

In accordance with the embodiments herein, the pet animal-wearable device further comprises one or more antennas for receiving and analyzing plurality of signals such as but not limited to Bluetooth Low Energy (BLE), Zigbee, ANT+, or other technologies to determine the associated pet animal status.

In accordance with the embodiments herein, the pet animal-wearable device further comprises one or more antennas for initiating a long-range communication with the cloud server. The antenna can include a SigFox radio or a Helium radio capable of communicating in ISM (Industrial, Scientific, and Medical) bands.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE ACCOMPANYING DRAWINGS

The other objects, features and advantages will occur to those skilled in the art from the following description of the preferred embodiment and the accompanying drawings in which:

FIG. 1 illustrates a block diagram of a pet animal-wearable device adopted for determining pet status/safety and tracking the location of the pet animal, according to one embodiment herein;

FIG. 2 is a flow diagram illustrating events (E_n) causing state transition and actions (A_n) taken at each state for managing safety of the pet animal, according to one embodiment herein;

FIG. 3 is a flow diagram illustrating a method for determining pet animal status/safety, communicating the pet animal status information to the pet animal owner and tracking the location of the pet animal according to one embodiment herein; and

FIG. 4 is a flow chart illustrating the steps involved in a method for identifying current status and current location of a pet animal, according to one embodiment herein.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed, description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a pet animal-wearable device for determining the status of the pet animal by leveraging wireless signal information gathered by the device over time. The time series of the signal information is analyzed to identify useful patterns that are used to determine the status of a pet animal. The pet animal status is a multi-dimensional, qualitative or quantitative indicator such as but not limited to a GPS Latitude/Longitude of the pet animal, safe or unsafe condition of the pet animal, etc. The sate condition is any of a condition selected from the group comprising, pet animal being at home, or easily reachable by the owner (or owner's friends and family members), or at a place that is confirmed by the owner to be safe. Examples of safe status include the pet animal is on a regular walk with the owner or a designated pet animal walker; or the pet animal is at a pet hotel confirmed by the owner. Further, the unsafe condition refers to a condition where the pet animal is at an unknown or inaccessible place for the owner.

In accordance with the embodiments herein, the status of the pet animal is determined by various factors/conditions such as but not limited to identifying whether the pet animal is in an enclosed space or not, identifying if the pet animal is in extreme temperature not suitable for the pet animal's health, identifying whether the pet animal is moving or stationary, identifying if the pet animal-wearable device/tag is removed, etc.

In accordance with the embodiments herein, the determined status of the pet animal is used for deferring the use of GPS and cellular techniques until pet animal has a high probability to be unsafe.

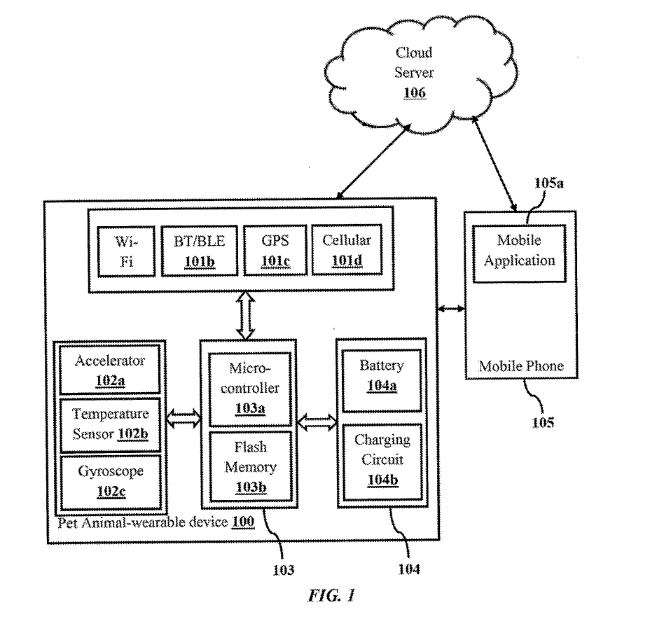

FIG. 1 illustrates a block diagram of a self learning monitoring device (referred to as a pet animal-wearable device, hereafter) adapted for determining pet animal status/safety and tracking the location of the pet animal, according to one embodiment. The pet animal-wearable device 100 comprises a plurality of radio components such as but not limited to a Wi-Fi radio 101a, a Bluetooth radio 101b, an embedded GPS 101c, a cellular communication module 101d, etc; a plurality of sensors such as font not limited to a light sensor (not shown in FIG. 1), a temperature sensor 102b, a motion sensor (not shown in FIG 1), an accelerometer sensor 102a, a gyroscope 102c, etc; a central processing unit 103 comprising a micro-controller 103a and a flash memory 103b; and a power management module 104 comprising a battery 104a and a battery charging circuit 104b.

In accordance with the embodiments herein, the Bluetooth radio 101b and Wi-Fi radio 101a of the pet animal-wearable device 100 are used to autonomously learn and infer pet animal status in order to defer the use of GPS 101c and the cellular communication module 101d, until pet animal has a high probability to be unsafe (e.g., far away from the home not accompanied by a family member or friend of the owner). Such deferred use of GPS 101c and cellular communication module 101d is critical in conserving the battery 104a power of the pet animal-wearable device.

In accordance with the embodiments herein, the Bluetooth radio 101b and the Wi-Fi radio 101a periodically receive Bluetooth and Wi-Fi signals around the pet animal, with a much lower power consumption compared to the GPS 101c and cellular communication module 101d. Information including MAC address of the signal source and the signal strength are extracted from the received signal. Further, the extracted information is stored in the flash memory of pet animal-wearable device with a timestamp.

In accordance with, the embodiments herein, the pet animal-wearable device 100 transmits the received Bluetooth and Wi-Fi signals from the Bluetooth radio 101b and the Wi-Fi radio 101a to a cloud server 106. Further, the pet animal-wearable device 100 and the cloud server 106 analyze the collected/extracted signals (Bluetooth, and Wi-Fi signals) to identify signal sources that are highly correlated to the pet animal status.

In accordance with the embodiments herein, the light sensor is adapted for identifying whether the pet animal is in an enclosed space or not; the temperature sensor 102b is adapted for identifying if the pet animal is in extreme temperature not suitable for the pet animal's health; the motion sensor is adapted for identifying whether the pet animal is moving or stationary; the accelerometer sensor is adapted for detecting magnitude and direction of pet animal's movement; and the gyroscope is adapted for measuring or maintaining orientation. Further, the output of the said plurality of sensors is used by the micro-controller 103a of the central processing unit 103 for determining the status of the pet animal.

In accordance with the embodiments herein, the Wi-Fi radio 101a identifies residential or business Wi-Fi access points (APs), as they broadcast beacons. Further, the first three octets in a MAC address are used as the Organizational Unique Identifier (OUI). Based on the OUI, the Wi-Fi radio 101a identifies popular brand names such as but not limited to IBM™ PCs, Samsung™ mobile phones and Apple™ tablets and smart phones.

In accordance with the embodiments herein, a mobile application 105a is configured in pet animal's owner's mobile phone 105 for receiving a confirmation about the status of the pet animal. The confirmation (e.g., via a mobile phone application 105a) is required to confirm the pet animal is in safe status. Further, the GPS Latitude/Longitude information is used to identify the type of the place and its surrounding signal footprint. The identified place and its surrounding signal footprint information are stored into the flash memory 103b of the pet animal-wearable device 100 and/or in the cloud server 106. The identified place and its surrounding signal footprint information are used to determine the location and status of the pet animal as well as other pet animals at the current time instance or in future.

In accordance with the embodiments herein, the central processing unit 103 of the pet animal-wearable device 100 learns the identified signals and the signal sources to infer or predict the pet animal status. Examples for pet animal status prediction include; (i) a regular walk is correlated to the disappearance of home and close neighbors' AP and appearance of APs in the broad neighborhood. Most of the time, the pet animal is constantly accompanied by the owner's mobile phone 105. The pet animal may also be accompanied by a family member's mobile phone or a designated pet animal walker's mobile phone. (ii) Accompanied by the owner's mobile but also observing many new signal sources indicating an occasional walk away from the neighborhood, e.g., walking in the mall or playing in a pet animal park. Again, pet animal may be accompanied in this case by a family member or a pet animal walker's mobile phone. (iii) The pet animal is dropped to a Veterinary physician or pet animal hotel that has been previously visited. The device will recognize the surrounding wireless signal footprint to infer the type of the place. Sometimes, a GPS location is considered to further confirm the pet animal is indeed at a place indicated by the signal footprint. More specific and contextual questions are sent to the user/owner for confirmation, such as “Is your pet animal at this Veterinary physician?” via SMS or through a dedicated mobile application 105a installed in the user's/owner's mobile phone 105.

In accordance with the embodiments herein, the above learning and analysis process iterates over time to develop an accurate model of signal patterns correlated to pet animal status.

In accordance with, the embodiments herein, the pet animal-wearable device 100 uploads data/information to the cloud server 106 using Wi-Fi 101a or Bluetooth connections 101b. The cloud server 106 aggregates the data/information from a large population of pet animals to build a database of Bluetooth and Wi-Fi devices that can be used to quickly infer pet animal status. For example, signal footprint of regular pet animal walkers in an area (such as parks, veterinary physicians and pet animal hotels, etc) can be disseminated to the pet animal-wearable devices 100 in the vicinity, so that the pet animal-wearable devices 100 recognize this pet animal walker in future. Similar option applies to signal footprint of regional Veterinary physicians and pet animal hotels.

In accordance with the embodiments herein, the cloud server 106 requests a geographic location server such as the Google™ Geo-location server for getting the GPS latitude/longitude information based on the Wi-Fi APs, to determine the location and status of the pet animal.

In accordance with the embodiments herein, the pet animal-wearable device 100 further comprises one or more antennas for receiving and analyzing plurality of signals such as but not limited to BLE, Zigbee, ANT+, or other technologies to determine the associated pet animal status.

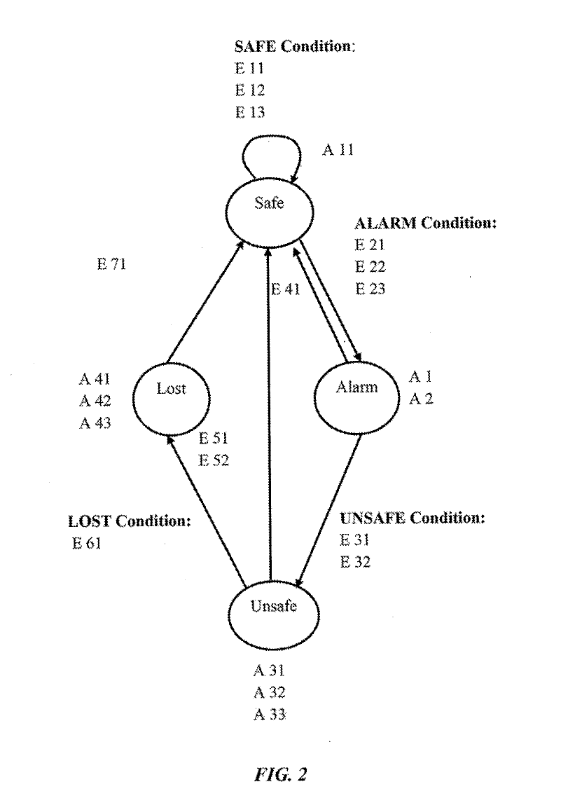

FIG. 2 is a flow diagram illustrating events (E_n) causing state transition and actions (A_n) taken at each state for managing safety of the pet animal, according to one embodiment. With respect to FIG. 2, the pet animal status is modeled using at-least four categories such as but not limited to a safe, alarm, unsafe and lost for determining the pet animal safety. Events (E_n) causing state transition and actions (A_n) taken at each state are as shown in FIG. 2. For events such as: E11—Pet animal on a regular walk with owner or designated pet animal walker, E12—Pet animal at home and E13—Pet animal playing in the backyard; then the action A11—Wi-Fi/BT scan at normal frequency determining the pet animal is “safe”. For events such as: E21—Pet animal walking in close neighborhood without owner or designated pet animal walker, E22—Pet animal goes to a dog park with owner, and E23—Pet animal goes to a shopping mall with owner; then the action A21—Wi-Fi/BT scans at increased frequency and initiates the “alarm” and further the A22—GPS is activated for retrieving Latitude/Longitude details if needed. Further, determining “safe”, when the event E41—Pet animal goes home, occurs. For events such as: E31—Pet animal runs into distant neighborhood, and E32—Pet animal left at veterinary physician or a pet animal hotel; then the action A31—Wi-Fi/BT scans at increased frequency, A32—GPS is activated for retrieving Latitude/Longitude details if needed, and A33—Sending an alert/distress signal/message to the user/owner about the “unsafe” location area for pet animal. Further, determining the location as “safe”, if user/owner appears or replies to the alert message (E52) and confirms the pet animal is safe (E51). Further, if the user/owner confirms the pet animal is “lost” (E61), then the action A41—GPS is activated for retrieving Latitude/Longitude details and sending the same to the user/owner, A42—Sending a distress signal/message embedded in Wi-Fi/BT beacons, and A43—notifying participating pet animal organization about the current location of the pet animal. Further, determining the pet animal is “safe”, when the pet animal is recovered (E71).

In accordance with the embodiments herein, the “safe” condition refers to: (a) Presence of owner's mobile devices in the vicinity, (b) Presence of mobile devices from owner's family members and close friends, (c) Presence of home Wi-Fi AP, Mix of (a) and/or (b) and/or (c) and other signal sources.

In accordance with the embodiments herein, the “alarm” condition refers to: presence of neighborhood Wi-Fi APs with regular timestamp, but without sources from the safest and safe categories, and Presence of mostly new sources and intermittent signal from sources in safest and safe categories.

In accordance with the embodiments herein, the “unsafe” condition refers to: Presence of neighborhood Wi-Fi APs with unusual timestamp, and Presence of completely new sources.

In accordance with the embodiments herein, the “lost” condition refers to: owner or owner-authorized person confirming the pet animal is lost

In accordance with the embodiments herein, when the pet animal is in “unsafe” condition i.e. when there is a presence of neighborhood Wi-Fi APs with unusual timestamp, presence of completely new sources, no signal footprint from mobile devices owned by the owner, owner's friends and family members such as when the pet animal is at a pet animal hotel; then the pet animal-wearable device will make a guess at the pet animal safety, based on its surrounding wireless signals, which may or may not be recognized by the pet animal-wearable device. When the pet animal-wearable device can infer the pet animal is at a pet animal hotel, it will still estimate the pet animal safety to be at a low level as there is no signal with high, safety coefficient around the pet animal. Since the pet animal-wearable device is unsure about the pet animal safety, it will send an alert to the owner. Further, since the pet animal-wearable device is equipped with a GPS interface, it will activate the GPS to obtain a Latitude/Longitude details. This is particularly critical if there is no signal for the pet animal-wearable device to infer the pet animal is at a pet animal hotel. Further, the Latitude/Longitude details can be used by the cloud server to infer the pet animal is at a pet animal hotel.

In accordance with the embodiments herein, the alert comprises the inferred pet animal location and status. For example, “Is your pet animal currently at a pet animal hotel?” or “URGENT: we cannot determine the status of your pet animal although we have its location at this “XXX” place! Please verify the pet animal is safe.” The alert is sent via either a Wi-Fi connection if there happens to be a free Wi-Fi access point, or using the cellular connection, or other long-range communication technologies working in ISM bands. Further, the user may respond to the alert, to confirm the pet animal is at a pet animal hotel and safe. In this case, the pet animal-wearable device records the signal from access points around the pet animal and ignores the signals from mobile devices, as they are likely to be noise.

In accordance with the embodiments herein, when the user/owner indicates he/she is unaware of specified location and status, which is a strong indication that the pet animal is lost and an immediate action from the owner/user is required to recover the pet animal. Similar actions are adapted if the pet animal is wandering in the neighborhood such as at a shopping mall, pet animal park, etc. If the pet animal is indeed lost, the pet animal-wearable device starts broadcasting a “distress” signal. For example, such a “distress” signal can be embedded in either the Bluetooth low energy beacon or the Wi-Fi beacon sent by the pet animal-wearable device. Further, people are allowed to download a mobile application that can be “opted in” to support findings of lost pet animals by searching for “distress” signals in the vicinity. This is particularly useful if the pet animal-wearable device is too low on battery to support regular GPS and cellular (of other long-range communication) operations. Further, the “distress” signal is broadcasted to local shelters or other animal rescue organizations participating in the recovery task. As soon as the pet animal status is determined to be lost or unsafe, an alert is sent to participating shelters that are close to the pet animal location.

In accordance with the embodiments herein, the Bluetooth and Wi-Fi radios autonomously learn and infer pet animal status. The method for learning the pet animal status comprises receiving signals from the owner's mobile device which is usually strong when it is received, and correlating the received signals to repeat daily events undertaken by the pet animal, such as pet animal walk in the morning/evening and close proximity with the pet after dinner, receiving signals from the mobile devices owned by other family members (and anyone else who lives at home), which are usually strong when they appear and correlating the received signal to the pet animal being at home or on a walk with a family member, receiving signals from the mobile devices owned by a designated pet animal walker, which are usually strong when they appear and correlating the received signal to the pet animal on a walk, receiving signal from the home Wi-Fi access point, which is usually strong and appears whenever the pet animal is at home, receiving signals from the Wi-Fi access points in the close neighborhood, which are relatively weak but still appear most of the time when the pet animal is at home or in close proximity of the home such as in the yard, receiving signals from the Wi-Fi access points in the broad neighborhood, which may appear a few times every day and often follows a rough temporal order during pet animal walks, receiving and storing signals from the mobile devices that show up randomly at home (such as from visitors), and receiving signals from mobile devices that show up regularly during a pet animal walk (such as from a friend or pet animal pal), and receiving signals from places occasionally visited by the pet animal, such as but not limited to shopping malls, veterinary physicians, pet animal hotels, and friend's home,

In accordance with the embodiments herein, the central processing unit of the pet animal-wearable device learns the identified signals and the signal sources to infer or predict the pet animal status. Examples for pet animal status prediction include: (i) a regular walk is correlated to the disappearance of home and close neighbors' Wi-Fi AP (Access Point) and appearance of Wi-Fi APs in the broad neighborhood. Most of the time, the pet animal is constantly accompanied by the owner's mobile phone. The pet animal may also be accompanied by a family member's mobile phone or a designated pet animal walker's mobile phone. (ii) Accompanied by the owner's mobile but also observing many new signal sources indicating an occasional walk away from the neighborhood, e.g., walking in the mall or playing in a pet animal park. Again, pet animal may be accompanied in this case by a family member or a pet animal walker's mobile phone. (iii) The pet animal is dropped to a Veterinary physician or pet animal hotel that has been previously visited. The device will recognize the surrounding wireless signal footprint to infer the type of the place. Sometimes, a GPS location is considered to further confirm the pet animal is indeed at a place indicated by the signal footprint. More specific and contextual questions are sent to the user/owner for condonation, such as “Is your pet animal at this Veterinary physician?” via SMS or through a dedicated mobile application installed in the user's/owner's mobile phone.

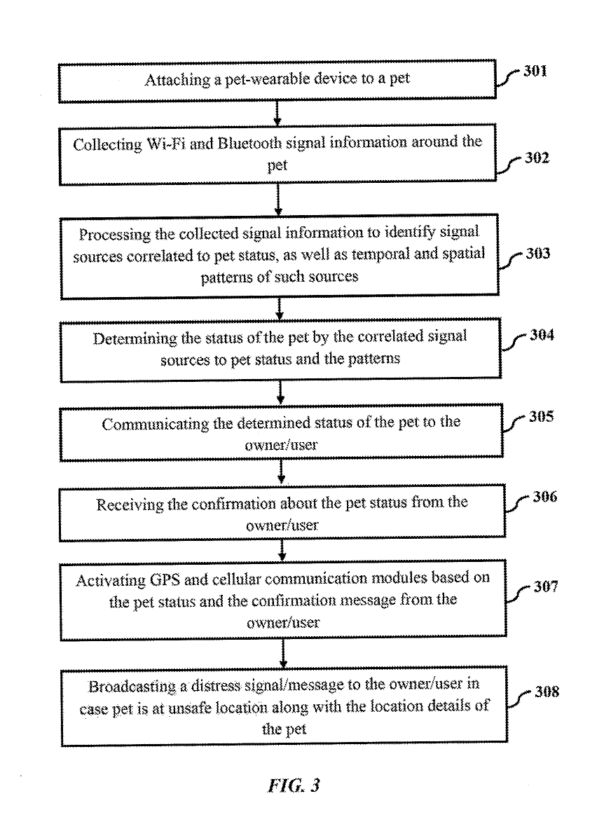

FIG. 3 is a flow diagram illustrating a method for determining pet animal status/safety, communicating the pet animal status information to the user/owner and tracking the location of the pet animal, according to one embodiment. The method for determining pet animal status/safety, communicating the pet animal status information to the user/owner and tracking the location of the pet animal comprises the steps of: attaching a pet animal-wearable device to the pet animal (Step-1), collecting Wi-Fi and Bluetooth signal information around the pet animal (Step-2), processing the collected signal information to identify signal sources correlated to pet animal status, as well as temporal and spatial patterns of such sources (Step-3), determining the status of the pet animal by the correlated signal sources to pet animal status and the patterns (Step-4), communicating the determined status of the pet animal to the owner/user (Step-5), receiving the confirmation about the pet animal status from the owner/user (Step-6), activating the GPS and cellular communication modules based on the pet animal status and the confirmation message from the owner/user (Step-7) and broadcasting distress signal/message to the owner/user in case pet animal is at unsafe location along with the location details of the pet animal (Step-8).

In accordance with the embodiments herein, the steps of determining the status of the pet animal further comprises analyzing the collected signal information both at an aggregated level, i.e. patterns across multiple pet animals are used for deriving the state of an individual pet animal and at individual level. The pet animal-wearable device autonomously infers/predicts the pet animal status as well as further enhances the predictions with the help of cloud server, based on the patterns specific to the pet animal.

In accordance with the embodiments herein, the central processing unit of the pet animal-wearable device activates the GPS and cellular communication modules (or other long-range communication modules in ISM bands) only if needed, i.e. the pet animal-wearable device is a user friendly and energy conserving pet animal tracking system.

In accordance with the embodiments herein, the GPS and cellular communication (or other long-range communication radio in ISM bands) remains deactivated when accompanied by owner's mobile device, even when the surrounding signal sources are new; GPS and cellular usage can be reduced to a minimum frequency.

In accordance with the embodiments herein, the central processing unit of the pet animal-wearable device activates the GPS and cellular communication modules (or other long-range communication modules in ISM bands), if the pet animal is wandering in the neighborhood without the owner (and hence the owner's mobile device) or without a family member.

In accordance with the embodiments herein, the central processing unit of the pet animal-wearable device activates the GPS and cellular communication modules (or other long-range communication modules in ISM bands), if the surrounding signal sources are completely new, which likely means the pet animal is lost. The pet animal-wearable device rates every signal source with a safety coefficient, which indicates how safe the pet animal is, if only that source is observed in the vicinity. A rule of thumb is, the safety coefficient increases if the source appears frequently in the past, and/or it appears with other sources of high safety coefficient. This helps in deferring the GPS and long-range communication usage if the pet animal is accompanied by signal sources with high safety coefficient. Further, the safety coefficient also decays over time. This helps to filter out noises picked up during occasional route or stay of the pet animals. For example, if a pet animal spends several days at a pet animal hotel, signals from other owners at the pet animal hotel or the workers at the pet animal hotel may have their safety coefficient boosted during the pet animal stay. But the safety coefficient of such signals quickly decay after the pet animal leaves the hotel. Similar method is applied to signals picked up from a pet animal park or a shopping mall.

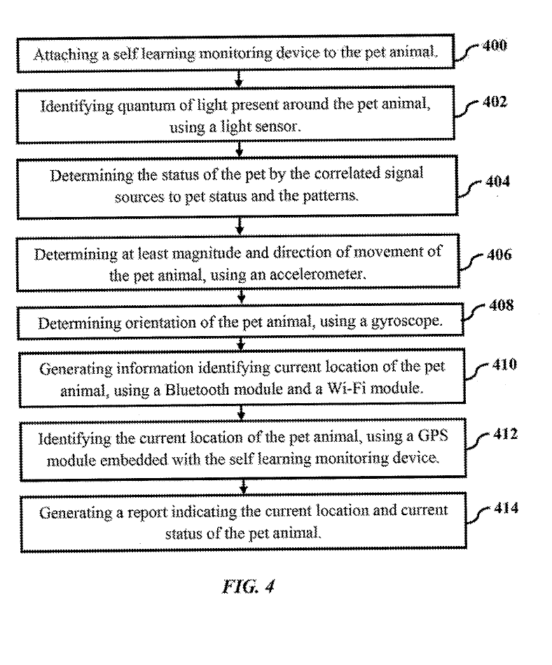

FIG. 4 is a flow diagram illustrating the steps involved in a method for identifying current status and current location of a pet animal. The method, in accordance with the preset disclosure includes the following steps:
attaching a self learning monitoring device to the pet animal whose current status and current location is to be determined (400);
identifying quantum of light present around the pet animal, using a light sensor embedded with the self learning monitoring device (402);
determining at least magnitude and direction of movement of the pet animal, using an accelerometer embedded with the self learning monitoring device (404);
determining orientation of the pet animal, using a gyroscope embedded with the self learning monitoring device (406);
generating: information identifying current location of the pet animal, using a Bluetooth module and a Wi-Fi module embedded with the self learning monitoring device (408);
identifying the current location of the pet animal, using a GPS module embedded with the self learning monitoring device, only in the event that the Bluetooth radio module and Wi-Fi radio module determine the pet animal wearing the device to be unsafe, and generate a trigger for activating the GPS module (410);
analyzing the quantum of light, magnitude and direction of movement, orientation and information corresponding to the current location of the pet animal (412); and
generating a report indicating the current location and current status of the pet animal (414).

In accordance with the embodiments herein, the method further includes the step of storing information, indicating the quantum of light, magnitude and direction of movement, orientation and current location of the pet animal, in a centralized server.

In accordance with the embodiments herein, the method further includes the step of creating at said centralized server, a listing of Bluetooth Devices and Wi-Fi devices that could be utilised to identify the current location of pet animals wearing self learning monitoring devices.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications.

It is also to be understood that the following claims are intended to cover all of the generic and specific features of the embodiments described herein and all the statements of the scope of the embodiments which as a matter of language might be said to fall there between.

We claim:

1. A self learning monitoring device configured to be attached to pet animals, said device configured to monitor at least current status and location of a pet animal wearing said device, said device comprising:
a plurality, of sensors embedded with said device, each of said plurality of sensors configured to determine a specific parameter corresponding to the pet animal wearing said device, said plurality of sensors including:
at least one light sensor configured to identity quantum of light present around the pet animal wearing said device;
at least one temperature sensor configured to determine temperature around the pet animal wearing said device;
at least one accelerometer configured to determine at least magnitude and direction of movement of the pet animal wearing said device;
at least one gyroscope configured to determine orientation of the pet animal wearing said device;
a Bluetooth radio module and a Wi-Fi radio module embedded with said device, said Bluetooth radio module and Wi-Fi radio module configured to generate information identifying the current location of the pet animal wearing the device;
a GPS module embedded with said device and configured to identify the current location of the pet animal wearing said device, said GPS module further configured to be activated by a trigger from the Bluetooth radio module and Wi-Fi radio module, when the pet animal wearing the device to determined to be unsafe;
a processing unit embedded with said device, said processing unit configured to analyze the specific parameter determined by each of said plurality of sensors, said processing unit further configured to analyze the information identifying the current location of the pet animal wearing the device, and generate a report indicating the current location and current status of the pet animal wearing the device.

2. The system as claimed in claim 1, wherein said plurality of sensors, said Bluetooth radio module and a Wi-Fi radio module, and said GPS module cooperate with a centralized server, said centralized server configured to store and collaborate the information received from said plurality of sensors, said Bluetooth radio module and a Wi-Fi radio module, and said GPS module.

3. The system as claimed in claim 1, wherein said accelerometer is a multi-axis accelerometer configured to detect and quantify the movements of the pet animal wearing said device.

4. The system as claimed in claim 2, wherein said centralized server is configured to create a relationship between a plurality of Bluetooth radio modules and Wi-Fi radio modules, and corresponding Bluetooth devices and Wi-Fi devices, based on the information exchanged there between, in respect of pet animals wearing said devices.

5. A method for identifying current status and current location of a pet animal, said method comprising the following steps:
attaching a self learning monitoring device to the pet animal whose current status and current location is to be determined;
identifying quantum of light present around the pet animal, using a light sensor embedded with the self learning monitoring device;
determining at least magnitude and direction of movement of the pet animal, using an accelerometer embedded with the self learning monitoring device;
determining orientation of the pet animal, using a gyroscope embedded with the self learning monitoring device;
generating information identifying current location of the pet animal, using a Bluetooth module and a Wi-Fi module embedded with the self learning monitoring device;
identifying the current location of the pet animal, using a GPS module embedded with the self learning monitoring device; and
analyzing the quantum of light, magnitude and direction of movement, orientation and information corresponding to the current location of the pet animal; and
generating a report indicating the current location and current status of the pet animal.

6. The method as claimed in claim 5, wherein said method further includes the step of storing information indicating the quantum of light, magnitude and direction of movement, orientation and current location of the pet animal, in a centralized server.

7. The method as claimed in claim 6, wherein said method further includes the step of creating at said centralized server, a listing of Bluetooth Devices and Wi-Fi devices that could be utilized to identify the current location of pet animals wearing self learning monitoring devices.
